(19) United States
(12) Patent Application Publication
(10) Pub. No.: US 2021/0151991 A1
(43) Pub. Date: May 20, 2021

(54) DEVICES AND METHODS FOR CHECKING BATTERY STATE OF HEALTH

(71) Applicant: Alcon Inc., Fribourg (CH)

(72) Inventors: Geoffrey C. Jawidzik, Mission Viejo, CA (US); Christopher Carl Jung, Mission Viejo, CA (US)

(21) Appl. No.: 17/092,756

(22) Filed: Nov. 9, 2020

Related U.S. Application Data
(60) Provisional application No. 62/935,862, filed on Nov. 15, 2019.

(51) Int. Cl. H02J 7/00; H02J 7/34; H02J 7/02; G01R 31/392; A61F 9/007

(52) U.S. Cl. CPC H02J 7/005; H02J 7/342; H02J 7/0013; A61B 2017/00734; G01R 31/392; A61F 9/007; H02J 7/02

(57) ABSTRACT

Devices and methods are disclosed for checking the state of health of rechargeable batteries. An electronic device comprises a plurality of rechargeable batteries and a controller, wherein the controller comprises a battery fuel gauge for performing a battery state of health check. The controller is configured to direct charge to connected loads and to transfer charge from the battery being checked to another battery during a battery state of health check. An example method comprises charging one battery to a full state, leaving another battery in a depleted state, and performing a state of health check on the battery in the full state while transferring charge via an intermediate controller from the battery in the full state to the battery in the depleted state.

### TECHNICAL FIELD

This disclosure is directed to devices and methods for checking the state of health of batteries.

### BACKGROUND

Rechargeable batteries are used in many different types of devices, such as mobile phones, electric vehicles, power tools, and medical devices, to name a few. As one example in the medical device field, ophthalmic surgical systems for performing ophthalmic surgical procedures such as cataract surgery and/or retinal surgery may have a console with a rechargeable battery and may also have a footswitch (i.e., foot controller) with a rechargeable battery. As an example, the battery in the console may be, for example, a lead-acid battery, and the battery in the footswitch may be, for example, a lithium-ion battery. The console typically has a power cord for receiving AC current from an outlet, and this current may be used to recharge the batteries. The footswitch may be connected or connectable to the console by a wire, in which case the wired connection enables recharging of the footswitch battery by power from the console. Additionally or alternatively, the footswitch may be operable wirelessly, and, when not in use, the footswitch may be mounted on the console adjacent a console panel that enables inductively recharging the footswitch battery by power from the console.

It can be useful to monitor certain characteristics of a battery and to provide information regarding those characteristics. For example, many rechargeable devices have electronics for monitoring the state of charge of the battery, i.e., the level of charge of the battery relative to its capacity. Electronics are also available for monitoring the state of health of a battery, i.e., the capacity of a battery relative to its original capacity. When a battery is used, its capacity can deteriorate over time. It can be useful to monitor a battery's state of health in order to know approximately how much useful life remains and to know when to replace the battery.

Commonly available electronic components used to determine battery state of health typically require that the battery be substantially discharged in a controlled manner during a state of health check. For example, certain electronic components for determining battery state of health require that the battery be discharged by about 30% of its capacity during state of health monitoring in order to accurately determine the battery's state of health. This discharging can produce heat which may require dissipation through use of a heatsink, whereby the energy of the discharging battery is dissipated through thermal convection to the environment. In some situations, the incorporation of such a heatsink may require undesirable technical and aesthetic compromises of the design. In addition, it may become necessary or desirable to use the device during a battery state of health check. If the state of health check is interrupted to use the device, the battery will have been discharged by some amount during the check, and the time that the device can be used before recharging is required therefore would have been reduced.

While prior ophthalmic surgical systems such as the CENTURION® Vision System and the like utilize a footswitch, the electrical design of the footswitch in such prior ophthalmic surgical systems has not included any means to check the state of health of the battery. The use of typical means for checking battery state of health in such a footswitch could result in drawbacks, such as undesirable technical and aesthetic compromises from the incorporation of a heatsink and/or operating with a depleted battery in the event the device is used during a state of health check. It would be desirable to be able to perform a battery state of health check in such footswitches and other devices without resulting in one or more of these drawbacks.

Accordingly, there is a continuing need for improved devices and methods for checking the state of health of batteries.

### SUMMARY

The present disclosure is directed to improved devices and methods for checking the state of health of batteries.

In some example embodiments, an electronic device, which may be for example a footswitch for an ophthalmic surgical system, is provided, the electronic device comprising: a plurality of batteries, wherein each battery of the plurality of batteries is rechargeable; and a controller, wherein each battery of the plurality of batteries is connected to the controller, and wherein the controller comprises a battery fuel gauge capable of performing a battery state of health check on each battery of the plurality of batteries. The controller is configured to direct charge to connected loads and to transfer charge from one battery of the plurality of batteries to another battery of the plurality of batteries during a battery state of health check. The controller may be configured to direct charge to connected loads from only one battery of the plurality of batteries at any given time.

In accordance with features of some example embodiments, each battery in the plurality of batteries may be a battery pack. The electronic device may be designed such that it does not include a heatsink for dissipating energy of a discharging battery through thermal convection. In embodiments in which the electronic device is a footswitch for an ophthalmic surgical system, the footswitch may be configured to communicate wirelessly with a console of the ophthalmic surgical system. The controller may be configured to inductively recharge one or more of the batteries of the plurality of batteries when the footswitch is placed on a charging station on the console.

The controller may be configured such that during a recharging of the electronic device, at least one battery is charged to a full state and at least one battery is left in a depleted state. The controller may be configured such that during the state of health check, the battery being checked is discharged by at least 20% of its capacity.

In some example embodiments, a method of checking battery state of health in an electronic device is provided, the method comprising: charging at least one battery of a plurality of batteries in the electronic device to a full state of electrical charge; leaving at least one other battery of the plurality of batteries in the electronic device in a depleted state of electrical charge; and performing a battery state of health check on a battery in the full state while transferring its charge via an intermediate controller to a battery in the depleted state. The method may further comprise separately checking the state of health of each of the batteries in the plurality of batteries by charging each battery, in turn, to a full state while leaving another battery in a depleted state and performing a battery state of health check on the battery in the full state while transferring charge from the battery in the full state to the battery in the depleted state.

The above examples and other examples will be understood by persons having ordinary skill in the art based on this disclosure.

### BRIEF DESCRIPTION OF THE DRAWINGS

The accompanying drawings illustrate examples of the devices and methods disclosed herein and, together with the description, serve to explain the principles of the disclosure.

FIG. 1 is a schematic diagram illustrating an example device in accordance with the disclosure.

FIG. 2 is a schematic diagram illustrating an example configuration for transferring charge between rechargeable batteries in accordance with the disclosure.

FIG. 3 is a flow chart showing steps in an example method in accordance with the disclosure.

The accompanying drawings may be better understood by reference to the following detailed description.

### DETAILED DESCRIPTION

For the purposes of explaining the principles of the disclosure, reference is made to the drawings, and specific language is used to describe the same. It will nevertheless be understood that, by reference to certain examples, no limitation of the scope of the disclosure is intended. Any alterations and further modifications to the described example systems, devices, instruments, and methods, and any further application of the principles of the disclosure, are fully contemplated as would normally occur to one skilled in the art to which the disclosure relates. In particular, the features, components, and/or steps described with respect to one example of the disclosure may be combined with features, components, and/or steps described with respect to other examples of the disclosure and may be modified and/or substituted as would normally occur to one skilled in the art. For simplicity, in some instances the same reference numbers may be used throughout the drawings to refer to the same or like parts.

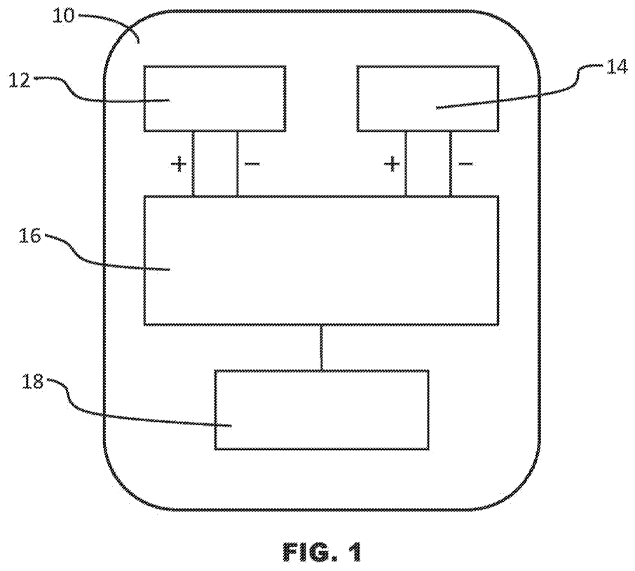

FIG. 1 is a schematic diagram illustrating one example device in accordance with the disclosure. The device is an electronic device 10, which is an electronic device that may be powered by rechargeable battery power.

As an example, electronic device 10 may be a footswitch that is part of an ophthalmic surgical system used for ophthalmic surgical procedures such as cataract surgery and/or retinal surgery. Except for differences as described herein, the ophthalmic surgical system may be similar to ophthalmic surgical systems as shown and described in U.S. Pat. No. 9,931,447, and/or to ophthalmic surgical systems that have been known and used, such as the CENTURION® Vision System available from Alcon Laboratories, Inc. (Fort Worth, Tex.) or the CONSTELLATION® Vision System available from Alcon Laboratories, Inc. (Fort Worth, Tex.), or any other ophthalmic surgical system suitable for use with the principles described herein. The ophthalmic surgical system may include an ophthalmic surgical console, which may comprise a housing and a fluidics cassette loaded into the housing. The ophthalmic surgical system may include the footswitch (an example of electronic device 10) to enable the operator to control certain functions of the ophthalmic surgical system by foot. For example, the footswitch may include one or more switches and/or buttons that can be actuated by foot. Actuation of the switches and/or buttons may enable scrolling through or switching between functions indicated on a display screen and/or may control such functions as the flow of fluidics, aspiration rate, phacoemulsification power, vitrectomy cut rate, intraocular lens injection rate, anterior capsulotomy, and/or coagulation power.

In accordance with the disclosure, electronic device 10 (e.g., a footswitch of an ophthalmic surgical system) comprises a plurality of batteries, i.e., at least two batteries, depicted schematically in FIG. 1 as a first battery 12 and a second battery 14. The term “battery” as used herein means a battery or battery pack, and the term “batteries” as used herein means plural batteries or battery packs. For example, each of the batteries 12, 14 in the electronic device 10 may be a battery pack, each of which may comprise more than one voltaic cell.

Electronic device 10 (e.g., a footswitch) further comprises a controller 16 that is electrically connected to each of the batteries 12, 14 in the plurality of batteries. Each battery 12, 14 in the plurality of batteries may be connected to the controller 16 via at least two conductors, one connected to the positive terminal of the battery and one connected to the negative terminal of the battery.

The controller 16 also connects via other electrical connections to electrical load 18, which schematically represents one or more connected loads corresponding to functionality requiring power. The load(s) 18 may be due to devices such as a motor that provides haptic feedback to the user, LEDs that provide visual indication of functional status to the user, a radio that communicates information to other devices, etc. In the example of the footswitch, the controller 16 comprises electronics for handling the functions of the footswitch. For example, the controller 16 may receive input from one or more switches and/or buttons on the footswitch and, in response to such input, may send signals to an ophthalmic surgical apparatus for performing the selected tasks or functions, such as one or more of the tasks or functions described above.

The electronic device 10 may be a wireless device, the batteries 12, 14 of which may be inductively rechargeable. For example, in the case of a footswitch associated with an ophthalmic surgical console, the console may have a power cord for receiving AC current from an outlet. When not in use, the footswitch may be placed on a charging station on the console adjacent a console panel that enables inductively recharging the footswitch batteries by power from the console. The controller 16 of the footswitch connects to the electrical power source from the ophthalmic surgical console when the footswitch is placed on its charging station to facilitate inductive recharging of the footswitch batteries.

In accordance with the disclosure, controller 16 also comprises electronics for performing a battery state of health check, such electronics for performing a battery state of health check referred to herein as a battery fuel gauge. Such a battery fuel gauge may comprise, for example, a fuel gauge integrated circuit such as those available from Texas Instruments Inc., or any other suitable electronics for checking battery state of health. Typically, such electronics used to determine battery state of health require that the battery being checked be substantially discharged in a controlled manner during the state of health check. For example, certain battery fuel gauges require that the battery be discharged by about 30% of its capacity during state of health monitoring in order to accurately determine the battery's state of health.

In certain prior devices with battery fuel gauges, this discharging of a battery during a state of health check could produce heat which would require dissipation through use of a heatsink, whereby the energy of the discharging battery would be dissipated through thermal convection to the environment. Also, in certain prior devices with battery fuel gauges, if the state of health check were to be interrupted to use the device, the battery would have been discharged by some amount during the state of health check, resulting in a somewhat depleted battery and, accordingly, a reduced time that the device could be used before requiring recharging.

In accordance with the disclosure, as described above, electronic device 10 (e.g., a footswitch) comprises a plurality of batteries 12, 14, and when it is desired to check the state of health of a first battery that is in a charged state, a second battery that is in a depleted state serves as a reservoir into which charge of the first battery is transferred during the check. The controller is configured such that when a state of health check is to be performed on a first battery that is in a charged state (e.g., battery 12 or 14) by the battery fuel gauge, the controller draws charge from the first battery and transfers it to a second battery that is in a depleted state (e.g., the other battery, battery 14 or 12). The battery fuel gauge performs the state of health check on the first battery during the transfer of charge from the first battery to the second battery.

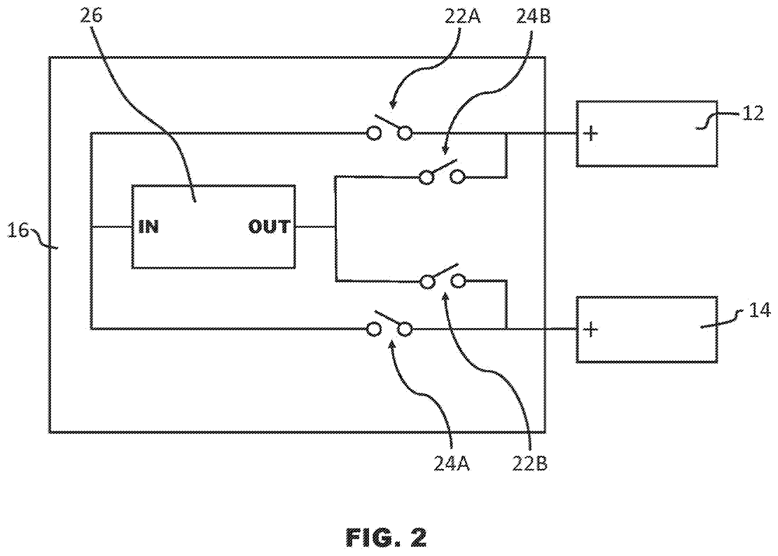

FIG. 2 is a schematic diagram illustrating an example configuration for transferring charge between rechargeable batteries in accordance with the disclosure. The controller 16 comprises a buck-boost charger 26 that can be selectively connected via switches to the rechargeable batteries 12 and 14. During a state of health check, the controller software enables the buck-boost charger 26 and enables a state for transferring charge from a full battery on which a state of health check is to be performed (e.g., battery 12 or 14) to a depleted battery (e.g., the other battery, battery 14 or 12). For example, when a state of health check is to be performed on battery 12, switches 22A and 22B are closed, while switches 24A and 24B are open. This enables transfer of charge from battery 12 through the buck-boost charger 26 to the battery 14. When a state of health check is to be performed on battery 14, switches 24A and 24B are closed, while switches 22A and 22B are open. This enables transfer of charge from battery 14 through the buck-boost charger 26 to the battery 12. If more than two rechargeable batteries are used, similar configurations may be used to enable a selective transfer of charge from any battery in the device to any other battery in the device.

In an example method of using the device illustrated in FIG. 1, before use of the electronic device 10 (e.g., footswitch), one battery of the plurality of batteries 12, 14 is charged to a “full” state of electrical charge (a “full” state of electrical charge may technically be less than actual full capacity, such as 70% of actual full capacity, to increase the service life of the battery), and another battery of the plurality of batteries 12, 14 is left at, or discharged to and then left at, a “depleted” state (a “depleted” state of electrical charge corresponds to a charge level that is sufficiently low so as to accept the charge to be transferred from the “full” battery during a state of health check and may technically be more charged than complete depletion, such as 30% of actual full capacity, to increase the service life of the battery). When the electronic device 10 (e.g., footswitch) is typically operated, the controller 16 directs power to be supplied from the charged battery 12 or 14 to the electrical load 18, as needed, until the user stops operating the electronic device 10 and returns it to its charging station, to be recharged.

Occasionally, as directed by the controller 16, upon reaching a “full” state of electrical charge, the “full” battery is run through a state of health check. During the state of health check, the “full” battery is automatically and purposefully discharged, with the electrical energy being transferred to the second, “depleted” battery, and with electrical parameters of the discharging being monitored by the battery fuel gauge as required to determine the state of health of the battery with required accuracy. During the state of health check, the battery being checked may be discharged by at least 20% of its capacity during state of health monitoring in order to accurately determine the battery's state of health. For example, during the state of health check, the battery being checked may be discharged by at least 25% of its capacity, by at least 30% of its capacity, by at least 35% of its capacity, or by at least 40% of its capacity during state of health monitoring. To increase the probability of this state of health check being successfully completed, by default the state of health check may be set to be done at times when the electronic device 10 (e.g., footswitch) is not normally in use.

If the electronic device 10 (e.g., footswitch) is put into use during the state of health check, i.e., before the state of health check is finished, then the state of health check is necessarily terminated prematurely, in which case neither battery involved in the transfer during the check is at a “full” state of electrical charge. However, the total charge shared between the two batteries is almost equal to that of one fully-charged battery, due to the high efficiency of the inter-battery charge transfer process. Immediately following such a terminated state of health check, the controller 16 may direct that power to be supplied to any electrical load(s) 18 be supplied first from whichever battery has a lower charge level, so as to reach as soon as possible a “depleted” charge state in one battery, which is a prerequisite for being able to perform a state of health check.

After an interrupted state of health check, upon returning the electronic device 10 (e.g., footswitch) to the charging station, one battery of the plurality of batteries 12, 14 is charged to a “full” state of electrical charge. When the electronic device is in its ready state for a state of health check (one battery “full” and one battery “depleted”), the controller 16 may reinitiate the state of health check.

Upon successful completion of a state of health check, the controller 16 directs that any power to be supplied to any electrical load 18 be supplied from the battery that is newly at a “full” state of electrical charge. The recharging process may be repeated, and the process of occasionally performing a successful state of health check, as described above, may be repeated at desired times or intervals indefinitely.

While FIG. 1 shows an example with two batteries 12, 14, the principles of the disclosure could be practiced with a device with more than two batteries, up to as many batteries as is practical for a particular application, with no more than half of the batteries being checked at any given time. The number of batteries to use in a particular application depends upon practical considerations such as electrical and mechanical design, cost, and other factors.

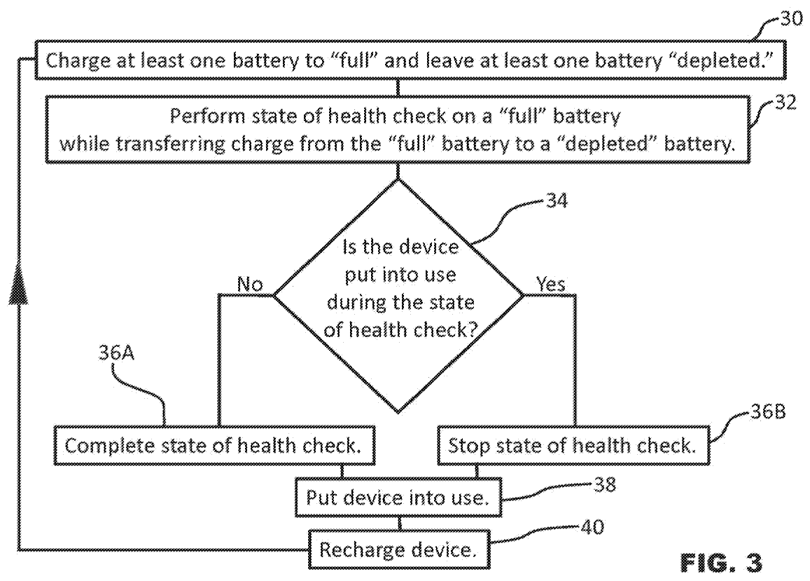

FIG. 3 is a flow chart showing steps in an example method in accordance with the disclosure. In step 30, before use of the electronic device 10 (e.g., footswitch), one battery of the plurality of batteries 12, 14 is charged to a “full” state of electrical charge (which may be less than actual full capacity), and another battery of the plurality of batteries 12, 14 is left at, or discharged to and then left at, a “depleted” state (which may technically be more charged than complete depletion). The electronic device 10 (e.g., footswitch) may be operated from this condition, in which case the controller 16 directs power to be supplied from the charged battery 12 or 14 to the electrical load(s) 18, as needed, until the user stops operating the electronic device 10 and recharges it, repeating step 30.

After completing step 30 with a “full” state of electrical charge for one battery, on selected occasions, as directed by the controller 16, step 32 is conducted, in which a state of health check is performed on the “full” battery. During the state of health check, the “full” battery is automatically and purposefully discharged, with the electrical energy being transferred to the “depleted” battery.

As indicated in flow chart element 34, if the electronic device (e.g., footswitch) is not put into use during the state of health check, then, as indicated in step 36A, the state of health check is completed. When the state of health check is completed, the battery that was “full” before the check is now depleted, and the battery that was “depleted” before the check is now full.

If the electronic device (e.g., footswitch) is thereafter put into use, as indicated at step 38, the controller 16 directs that any power to be supplied to any electrical load(s) 18 be supplied from the battery that is newly at a “full” state of electrical charge. As indicated at step 40, the recharging process may be repeated, at step 30. Which battery is charged at successive recharging operations may be performed based on any desired criteria or in any desired sequence. For example, the controller 16 may select that the battery that has the most charge (if any) be the one that is charged to “full.” Alternatively, if all batteries are “depleted,” the controller 16 may alternate which battery is charged to “full” upon successive recharging steps. Other battery charging sequences may be used. The steps of the flow chart of FIG. 3 may be repeated, with additional battery state of health checks being performed as desired on any battery or batteries that is or are “full” after step 30.

At flow chart element 34, if the electronic device (e.g., footswitch) is put into use during the state of health check, then, as indicated in step 36B, the state of health check is stopped without being completed. In this case, neither battery involved in the transfer during the check is at a “full” state of electrical charge. However, the total charge shared between the two batteries is almost equal to that of one fully-charged battery. In putting the electronic device (e.g., footswitch) into use, as indicated at step 38, the controller 16 may direct that power to be supplied to any electrical load 18 be supplied first from whichever battery has a lower charge level, so as to reach as soon as possible a “depleted” charge state in one battery. Power may thereafter be supplied from the other battery. Alternative sequencing of the battery from which to supply power may be employed. As indicated at step 40, the recharging process may be repeated, at step 30. As mentioned above, which battery is charged at successive recharging operations may be performed based on any desired criteria or in any desired sequence. The steps of the flow chart of FIG. 3 may be repeated, with additional battery state of health checks being performed as desired on any battery or batteries that is or are “full” after step 30.

Persons having ordinary skill in the art will recognize that devices and methods as disclosed herein have one or more advantages over prior approaches. For example, in comparison with prior approaches, devices and methods as disclosed herein avoid the need to use a heatsink, whereby the energy of the discharging battery is dissipated through thermal convection to the environment. In some situations, the incorporation of such a heatsink may require undesirable technical and aesthetic compromises of the design. For example, a footswitch of an ophthalmic surgical system or another electronic device may be desired or required to be water-tight (for example, to facilitate cleaning and/or for compliance with an IPX7 or IPX8 rating) and/or resistant to certain chemical cleaning agents or other solutions (e.g., balanced salt solutions). A footswitch of an ophthalmic surgical system or another electronic device may have a housing made of injection-molded plastic with a minimum number and size of seals. If a heatsink were required, an efficient heatsink would be made of metal and exposed on the exterior of the footswitch or other device, which would require the addition of a large, sealed opening in the housing. Thus, the use of a heatsink could compromise the water-tight characteristics or the chemical or solution resistance of the footswitch or other device. The use of a heatsink also could have a detrimental effect on the appearance of the footswitch or other device. Devices and methods as disclosed herein do not require a heatsink and thus can avoid these functional and aesthetic disadvantages.

In addition or alternatively, in comparison with prior approaches, devices and methods as disclosed herein avoid issues of prior devices that had the potential for significantly reduced useful life before recharging. For example, in certain prior devices, if the device were to be used during a state of health check, which use therefore interrupted the state of health check, the battery being checked will have been somewhat depleted during the check, leaving a reduced useful life of the battery before recharging. In other words, if the check was interrupted to use the device, the time of use before recharging is required would be reduced. With devices and methods as disclosed herein, the charge from one battery is transferred to another battery of the device during the state of health check. In some embodiments, the total charge shared between the two batteries is almost equal to that of one fully-charged battery. Thus, if the device were to be used during a state of health check, the time that the device could be used before recharging is approximately the same as it would have been if the state of health check had not been performed. A device in accordance with the disclosure may be designed so that, during or after a state of health check, the device does not have any state in which there is insufficient total stored electrical charge to meet the device's run-time requirement.

Persons of ordinary skill in the art will appreciate that the implementations encompassed by the disclosure are not limited to the particular exemplary implementations described above. In that regard, although illustrative implementations have been shown and described, a wide range of modification, change, and substitution is contemplated in the foregoing disclosure. It is understood that such variations may be made to the foregoing without departing from the scope of the disclosure. Accordingly, it is appropriate that the appended claims be construed broadly and in a manner consistent with the disclosure.

What is claimed is:

1. A footswitch for an ophthalmic surgical system, the footswitch comprising:
a plurality of batteries, wherein each battery of the plurality of batteries is rechargeable; and
a controller, wherein each battery of the plurality of batteries is connected to the controller, and wherein the controller comprises a battery fuel gauge capable of performing a battery state of health check on each battery of the plurality of batteries;
wherein the controller is configured to direct charge to connected loads and to transfer charge from one battery of the plurality of batteries to another battery of the plurality of batteries during a battery state of health check.

2. A footswitch for an ophthalmic surgical system as in claim 1, wherein each battery in the plurality of batteries is a battery pack.

3. A footswitch for an ophthalmic surgical system as in claim 1, wherein the footswitch does not include a heatsink for dissipating energy of a discharging battery through thermal convection.

4. A footswitch for an ophthalmic surgical system as in claim 1, wherein the footswitch is configured to communicate wirelessly with a console of the ophthalmic surgical system.

5. A footswitch for an ophthalmic surgical system as in claim 4, wherein the controller is configured to inductively recharge one or more of the batteries of the plurality of batteries when the footswitch is placed on a charging station on the console.

6. A footswitch for an ophthalmic surgical system as in claim 1, wherein the controller is configured such that during a recharging of the footswitch, at least one battery is charged to a full state and at least one battery is left in a depleted state.

7. A footswitch for an ophthalmic surgical system as in claim 1, wherein the controller is configured such that during the state of health check, the battery being checked is discharged by at least 20% of its capacity.

8. A footswitch for an ophthalmic surgical system as in claim 1, wherein the controller is configured to direct charge to connected loads from only one battery of the plurality of batteries at any given time.

9. An electronic device with rechargeable batteries and electronics for checking the state of health of the rechargeable batteries, the electronic device comprising:
a plurality of batteries, wherein each battery of the plurality of batteries is rechargeable; and
a controller, wherein each battery of the plurality of batteries is connected to the controller, and wherein the controller comprises a battery fuel gauge capable of performing a battery state of health check on each battery of the plurality of batteries;
wherein the controller is configured to direct charge to connected loads and to transfer charge from one battery of the plurality of batteries to another battery of the plurality of batteries during a battery state of health check.

10. An electronic device as in claim 9, wherein each battery in the plurality of batteries is a battery pack.

11. An electronic device as in claim 9, wherein the electronic device does not include a heatsink for dissipating energy of a discharging battery through thermal convection.

12. An electronic device as in claim 9, wherein the controller is configured to inductively recharge one or more of the batteries of the plurality of batteries when the electronic device is placed on a charging station.

13. An electronic device as in claim 9, wherein the controller is configured such that during a recharging of the electronic device, at least one battery is charged to a full state and at least one battery is left in a depleted state.

14. An electronic device as in claim 9, wherein the controller is configured such that during the state of health check, the battery being checked is discharged by at least 20% of its capacity.

15. An electronic device as in claim 9, wherein the controller is configured to direct charge to connected loads from only one battery of the plurality of batteries at any given time.

16. A method of checking battery state of health in an electronic device, the method comprising:
charging at least one battery of a plurality of batteries in the electronic device to a full state of electrical charge;
leaving at least one other battery of the plurality of batteries in the electronic device in a depleted state of electrical charge; and
performing a battery state of health check on a battery in the full state while transferring its charge via an intermediate controller to a battery in the depleted state.

17. A method of checking battery state of health in an electronic device as in claim 16, wherein each battery in the plurality of batteries is a battery pack.

18. A method of checking battery state of health in an electronic device as in claim 16, wherein the electronic device does not include a heatsink for dissipating energy of a discharging battery through thermal convection.

19. A method of checking battery state of health in an electronic device as in claim 16, further comprising separately checking the state of health of each of the batteries in the plurality of batteries by charging each battery, in turn, to a full state while leaving at least one other battery in a depleted state and performing a battery state of health check on the battery in the full state while transferring charge via the intermediate controller from the battery in the full state to the battery in the depleted state.

20. A method of checking battery state of health in an electronic device as in claim 16, wherein the controller is configured such that during the state of health check, the battery being checked is discharged by at least 20% of its capacity.
